(19) United States
(12) Patent Application Publication
(10) Pub. No.: US 2026/0174809 A1
(43) Pub. Date: Jun. 25, 2026

(54) MODULATION OF THE GUT MICROBIOME FOR ENHANCEMENT OF GUT AND BRAIN HEALTH

(71) Applicants: The Cleveland Clinic Foundation, Cleveland, OH (US); Standard Process, Palmyra, WI (US)

(72) Inventors: Gail CRESCI, Cleveland, OH (US); John Troup, Palmyra, WI (US)

(21) Appl. No.: 19/127,888

(22) PCT Filed: Nov. 8, 2023
(86) PCT No.: PCT/US2023/079116

§ 371 Date: May 7, 2025

Related U.S. Application Data
(60) Provisional application No. 63/382,933, filed on Nov. 9, 2022.

(51) Int. Cl. A61K 35/747; A61K 9/00; A61K 9/16; A61P 25/28

(52) U.S. Cl. CPC A61K 35/747; A61K 9/0019; A61K 9/1623; A61K 9/1652; A61K 9/1664; A61P 25/28

(57) ABSTRACT

The present invention provides compositions, systems, and methods for treating or preventing brain disorder with compositions comprising a prebiotic (e.g., green banana flour) and one or both of Lactobacillus reuteri, and Lactobacillus plantarum. In certain embodiments, the brain disorder is selected from neuroinflammation, a neurological disorder, a neuropsychiatric disorder, and a behavioral disorder. In some embodiments, the prebiotic, Lactobacillus reuteri, and Lactobacillus plantarum are provided as a powder inside an ingestible capsule.

The present application claims priority to U.S. Provisional application Ser. No. 63/382,933, filed Nov. 9, 2022, which is herein incorporated by reference in its entirety.

### FIELD OF THE INVENTION

The present invention provides compositions, systems, and methods for treating or preventing brain disorder by targeting the gut microbiota with compositions comprising a prebiotic (e.g., green banana flour) and one or both of Lactobacillus reuteri, and Lactobacillus plantarum.In certain embodiments, the brain disorder is selected from neuroinflammation, a neurological disorder, a neuropsychiatric disorder, and a behavioral disorder. In some embodiments, the prebiotic, Lactobacillus reuteri, and Lactobacillus plantarum are provided as a powder inside an ingestible capsule.

### BACKGROUND

A bidirectional communication exists between the gut microbiome and the brain, commonly known as the gut-brain axis. Alterations in gut microbial composition have been associated with many neurological, neuropsychiatric, and behavioral disorders. The gut microbiota contributes to homeostasis and behavior in the “host” through both direct and indirect crosstalk with the nervous system. The routes of communication involve the autonomic nervous system (e.g., enteric nervous system (ENS) and the vagus nerve), the neuroendocrine system, the hypothalamic-pituitary-adrenal (HPA) axis, the immune system and metabolic pathways.

Within the gut, the microbiota produce neuroactive compounds such as neurotransmitters (e.g., γ-aminobutyric acid (GABA), noradrenaline, dopamine, and serotonin (5-hydroxytryptamine (5-HT)), amino acids (e.g., tyramine, tryptophan), and microbial metabolites (e. g, short chain fatty acids and 4-ethylphenylsulfate). Through the portal circulation, these metabolites can interact with the host immune system, influence metabolism, and affect local neuronal cells of the enteric nervous system and afferent pathways of the vagus nerve that signal directly to the brain. The gut microbiota can influence the gut barrier integrity to control passage of signaling molecules to the intestinal lamina propria, which contains immune cells and terminal ends of ENS neurons, or to the portal circulation.

Many neuropsychiatric and behavior disorders (e.g., anxiety, depression, autism spectrum disorder) are associated with gut dysbiosis and impaired gut integrity. Stress can activate the HPA axis, involving neurons of the hypothalamus that secrete hormones into the brain or portal system, triggering the release of adrenocorticotrophic hormone (ACTH) which initiates the synthesis and secretion of cortisol. Cortisol regulates neuroimmune signaling responses that then impact the gut barrier integrity. Stress hormones, immune mediators, and CNS neurotransmitters can activate neuronal cells of the ENS and afferent pathways of the vagus nerve which can alter the gut environment and the gut microbiota composition.

### SUMMARY OF THE INVENTION

The present invention provides compositions, systems, and methods for treating or preventing brain disorder by targeting the gut microbiota with compositions comprising a prebiotic (e.g., green banana flour) and one or both of Lactobacillus reuteri, and Lactobacillus plantarum (psychobiotics). In certain embodiments, the brain disorder is selected from neuroinflammation, a neurological disorder, a neuropsychiatric disorder, and a behavioral disorder. In some embodiments, the prebiotic, Lactobacillus reuteri, and Lactobacillus plantarum are provided as a powder inside an ingestible capsule.

In particular embodiments, provided herein are systems comprising: a) an ingestible capsule; b) a prebiotic (e.g., that provides a carbon source); c) at least one bacteria selected from: i) Lactobacillus reuteri, and ii) Lactobacillus plantarum.

In certain embodiments, the at least one bacteria comprises both the Lactobacillus reuteri, and Lactobacillus plantarum.In other embodiments, the at least one bacteria comprises Lactobacillus reuteri, and the Lactobacillus reuteri comprises Lactobacillus reuteri 3613. In some embodiments, the at least one bacteria comprises Lactobacillus plantarum, and the Lactobacillus plantarum comprises Lactobacillus plantarum 276.

In particular embodiments, the at least one bacteria and the prebiotic are present inside the capsule. In further embodiments, the capsule comprises a vegetable-based capsule or a gelatin-based capsule.

In other embodiments, the at least one bacteria and the prebiotic are mixed and in the form of a powder. In further embodiments, the powder is present in the capsule. In additional embodiments, the prebiotic comprises resistant starch. In some embodiments, the resistant starch is selected from the group consisting of: green banana flour, potato starch, and rolled oats. In particular embodiments, the prebiotic comprises NUBANA RS65G Green Banana Flour. In other embodiments, the prebiotic comprises oligosaccharides. In particular embodiments, the oligosaccharides are selected from the group consisting of: galactooligosaccharides, fructooligosaccharides, and human milk oligosaccharides.

In further embodiments, the at least one bacteria and the prebiotic are present inside the capsule, and wherein 2-15 mg (e.g., about 2, 3, 4, 5 , 6, 7, 8, 9, 10, 11, 12, 13, 14, or 15 mg) of the at least one bacteria are present in the capsule. In other embodiments, 20-700 mg (e.g., about 20 . . . 40 . . . 60 . . . 100 . . . 150 . . . 300 . . . 450 . . . 600 . . . or 700 mg) of the prebiotic is present in the capsule.

In certain embodiments, provided herein are methods of treating or preventing a brain disorder comprising: administering a composition to a subject, or providing the composition to the subject such that the subject administers the composition to themselves, wherein the composition comprises: a) a prebiotic (e.g., that provides a carbon source), and b) at least one bacteria selected from Lactobacillus reuteri, and Lactobacillus plantarum, and wherein the subject has, or is at risk of, a brain disorder.

In some embodiments, the brain disorder is selected from the group consisting of: neuroinflammation, a neurological disorder, a neuropsychiatric disorder, and a behavioral disorder. In further embodiments, the brain disorder is selected from the group consisting of: alcohol use disorder, drug addiction, depression, anxiety, and Alzheimer's disease.

In certain embodiments, the composition is in the form of a tablet. In other embodiments, the composition is present inside a suppository. In further embodiments, the composition is present inside a capsule. In further embodiments, the composition is present the composition is included in a chewable gummy. In further embodiments, the composition is present in a food or beverage (e.g., yogurt, yogurt drink). In particular embodiments, the administering or administers is orally. In further embodiments, the administering or administer is rectally.

In some embodiments, the at least one bacteria comprises both the Lactobacillus reuteri, and Lactobacillus plantarum.In further embodiments, the at least one bacteria comprises Lactobacillus reuteri, and the Lactobacillus reuteri comprises Lactobacillus reuteri 3613. In further embodiments, the at least one bacteria comprises Lactobacillus plantarum, and the Lactobacillus plantarum comprises Lactobacillus plantarum 276. In additional embodiments, the at least one bacteria and the prebiotic are present inside a capsule. In certain embodiments, the capsule comprises a vegetable-based capsule. In other embodiments, the capsule comprises a gelatin-based capsule. In some embodiments, the at least one bacteria and the prebiotic are mixed and in the form of a powder. In other embodiments, the powder is present in a capsule.

In some embodiments, the prebiotic comprises resistant starch. In other embodiments, the resistant starch is selected from the group consisting of: green banana flour, potato starch, and rolled oats. In particular embodiments, the prebiotic comprises NUBANA RS65G Green Banana Flour. In additional embodiments, the prebiotic comprises oligosaccharides. In some embodiments, the oligosaccharides are selected from the group consisting of: galactooligosaccharides, fructooligosaccharides, and human milk oligosaccharides.

In additional embodiments, at least part of the composition is present inside a capsule, and wherein 2-15 mg of the at least one bacteria are present in the capsule. In further embodiments, 20-700 mg of the prebiotic is present in the capsule. In some embodiments, at least part of the composition is present inside four to six capsules, and wherein 2-15 mg (e.g., about 2, 3, 4, 5, 6, 7, 8, 9, 10, 11, 12, 13, 14, or 15 mg) of the at least one bacteria is present in each of the four to six capsules. In further embodiments, 20-700 mg (e.g., about 20 . . . 40 . . . 60 . . . 100 . . . 150 . . . 300 . . . 450 . . . 600 . . . or 700 mg) of the prebiotic is present in each of the four to six capsules.

In further embodiments, the administering or the administers provides the subject with the four to six capsules on a first day. In some embodiments, the administering or the administers is repeated on a second day. In other embodiments, the administering or the administers is repeated for 3 . . . 6 . . . 30 . . . 180 . . . 365 days or more. In additional embodiments, the subject is a human (e.g., a human with anxiety, alcohol dependence, or other disease).

In some embodiments, provided herein are formulations for oral administration to a subject, the formulation comprising isolated strains Lactobacillus reuteri and Lactobacillus plantarum, and a prebiotic (e.g., that provides a carbon source), wherein the formulation is in the form of a powder.

In certain embodiments, the formulation is in the form of a capsule, a tablet, a chewy, a sachet, or a food product. In some embodiments, the formulation comprises an ingestible carrier. In other embodiments, the ingestible carrier comprises a dairy product. In some embodiments, the ingestible carrier comprises a dressing or a beverage. In further embodiments, the ingestible carrier comprises acidified milk, yogurt, frozen yogurt, milk powder, milk concentrate, or cheese. In further embodiments, the Lactobacillus reuteri comprises Lactobacillus reuteri 3613. In some embodiments, the Lactobacillus plantarum comprises Lactobacillus plantarum 276. In additional embodiments, the prebiotic comprises resistant starch. In other embodiments, the resistant starch is selected from the group consisting of: green banana flour, potato starch, and rolled oats. In certain embodiments, the prebiotic comprises NUBANA RS65G Green Banana Flour. In other embodiments, the prebiotic comprises oligosaccharides. In additional embodiments, the oligosaccharides are selected from the group consisting of: galactooligosaccharides, fructooligosaccharides, and human milk oligosaccharides.

In some embodiments, provided herein is a foodstuff comprising the formulations or compositions described herein.

### DESCRIPTION OF FIGURES

FIG. 1 shows body weight changes from Example 1 below. All mice tolerated the treatments and supplements well. All mice demonstrated expected weight gain and there were no differences in body weight changes between the groups.

FIG. 2 shows the cecum weight-to-body weight results from Example 1 below. Germ-free and/or antibiotic treated (dysbiotic) mice phenotypically have an enlarged cecum. Therefore, we measured the weight of mouse cecum relative to body weight as a surrogate measure for gut dysbiosis. As expected, we found cecum-to-body weight to be highest in the ethanol-saline exposed mice. Mice supplemented with the probiotic blend or synbiotic had a reduced cecum: BW ratio (*P<0.05), similar to chow-fed mice.

FIG. 3 shows gram negative bacteria in cecum as described in Example 1. Ethanol exposure is known to deplete phyla Bacteroidetes and Firmicutes and induce Proteobacteria, which contains gram negative bacteria. Here we tested for overgrowth of gram negative bacteria by selective agar plating with mouse cecal contents. We found that the pair-fed mice, a diet that is high in fat and low in fiber, had the highest presence of gram negative bacteria followed by the ethanol-saline exposed mice. The mice supplemented with probiotic blend or synbiotic had the lowest presence of gram negative bacteria in the cecum (p=0.08).

FIG. 4 shows liver triglyceride levels as described in Example 1 below. The ethanol-feeding model induces hepatic steatosis, therefore we looked for differences in liver triglyceride between the treatment groups. We found that ethanol induced liver triglyceride levels compared to the chow-fed and pair-fed mice, but ethanol-exposed mice supplemented with the synbiotic had significantly lower liver triglyceride levels compared to the ethanol-saline treated mice (*P<0.05). FIG. 5 shows the mRNA expression of two tight junctional proteins, occludin and zonulen occluden (ZO-1) in the proximal colon as described in Example 1. Both occludin and ZO-1 mRNA relative expression was significantly higher in the ethanol exposed mice supplemented with the synbiotic (*p<0.05).

FIG. 6 shows staining results for ZO-1 and Occludin in proximal colon in mice as described in Example 1. It was found that ethanol-exposed mice treated with the probiotic blend (EF-pro) had increased staining intensity for ZO-1 (green) and Occludin (red) in the proximal colon compared to mice exposed to ethanol/saline (EF-S).

FIG. 7 shows results from Example 1, which show the tested probiotic/synbiotic supplementation in ethanol treated mice increased mRNA expression levels of superoxide dismutase 2(SOD2 ). This is important as ethanol metabolism is known to induce reactive oxygen species and cause oxidative stress, while SOD2 clears mitochondrial reactive oxygen species (ROS) and thus plays an anti-apoptotic role against oxidative stress. We tested for the mRNA expression of SOD2 in the proximal colon and found that the synbiotic induced SOD2 during ethanol exposure compared to the ethanol-saline group (*p<0.05).

FIG. 8A shows results from Example 1, which shows CNS exposure to ethanol deregulates neuroimmune signaling triggering microglial activation. FIG. 8A data shows chronic-binge ethanol exposure induced microglial activation (ionized calcium binding adaptor molecule 1; Iba1) in mice, which was mitigated in mice supplemented daily with a neurotransmitter-targeted synbiotic. FIG. 8B shows Iba1 (green) intensity staining semi-quantification. *p<0.05.

FIG. 9. It is known that, after becoming activated, microglia produce and secrete a plethora of inflammatory molecules that when in excess in the parenchyma further contributes to neuronal damage. Ethanol-induced microglial activation (FIG. 8) coincided with increased mRNA expression of proinflammatory cytokines and chemokine, and synbiotic supplementation protected against this occurrence, as shown in this figure. *p<0.05.

### DEFINITIONS

A “subject” is an animal such as vertebrate, preferably a domestic animal or a mammal.

Mammals are understood to include, but are not limited to, murines, simians, humans, bovines, cervids, equines, porcines, canines, felines etc.

“Administration” refers to the delivery of one or more agents to a subject. In some embodiments, compositions described herein are delivered orally. In some embodiments, compositions are delivered rectally or through another suitable delivery method.

As used herein, the terms “resistant starch” and “RS” refer to any starch that is not digested in the small intestine but passes to the large bowel. “Resistant starch” includes naturally occurring resistant starches and non-resistant starches that are made resistant through a manufacturing process (e.g., by encapsulation, or by chemical modification or other means). The term includes starches that are partially resistant but processed to increase the RS fraction, and processes that produce RS unintentionally (e.g., not engineered specifically to product RS). In some embodiments, RS pass through the intestines completely undigested. In some embodiments, RS are digested very slowly or incompletely.

### DETAILED DESCRIPTION

The present invention provides compositions, systems, and methods for treating or preventing brain disorder with compositions comprising a prebiotic (e.g., green banana flour) and one or both of Lactobacillus reuteri, and Lactobacillus plantarum.In certain embodiments, the brain disorder is selected from neuroinflammation, a neurological disorder, a neuropsychiatric disorder, and a behavioral disorder. In some embodiments, the prebiotic, Lactobacillus reuteri, and Lactobacillus plantarum are provided as a powder inside an ingestible capsule.

In some embodiments, the prebiotic, Lactobacillus reuteri, and Lactobacillus plantarum are formulated for oral administration. The present disclosure is not limited to particular methods of oral administration. Examples include, but are not limited to, food products, foods, nutraceuticals, nutritional supplements, capsules, etc. In some embodiments, the prebiotic, Lactobacillus reuteri, and Lactobacillus plantarum are encapsulated. In some embodiments, a capsule shell that is constructed to dissolve at a predetermined pH of a target region (e.g., large intestine, small intestine, bowel, etc.) is utilized (e.g., available from Assembly Biosciences, Carmel, IN). In some embodiments, capsules also have inner and outer layers that can be engineered to dissolve at different pH levels, making it possible to use a single capsule to deliver two doses of a therapeutic to different locations in the GI tract, or to deliver two different therapeutics to different locations. In some embodiments, mucin is used in the encapsulation technology to protect against stomach acid.

In some embodiments, the Lactobacillus reuteri, and Lactobacillus plantarum are provided in a single or separate capsules. In some embodiments, the prebiotic is provided separately. In some compositions embodiments, compositions are formulated in pharmaceutical compositions. The bacteria of embodiments of the disclosure may be administered alone or in combination with pharmaceutically acceptable carriers or diluents, and such administration may be carried out in single or multiple doses.

Compositions may, for example, be in the form of tablets, resolvable tablets, capsules, bolus, drench, pills sachets, vials, hard or soft capsules, aqueous or oily suspensions, aqueous or oily solutions, emulsions, powders, granules, syrups, elixirs, lozenges, reconstitutable powders, liquid preparations, creams, troches, hard candies, sprays, chewing-gums, creams, salves, jellies, gels, pastes, toothpastes, rinses, dental floss and tooth-picks, liquid aerosols, dry powder formulations, HFA aerosols or organic or inorganic acid addition salts.

The pharmaceutical compositions of embodiments of the disclosure may be in a form suitable for oral or rectal administration. Depending upon the disorder and patient to be treated and the route of administration, the compositions may be administered at varying doses.

For oral administration, bacteria of embodiments of the present disclosure may be combined with various excipients. Solid pharmaceutical preparations for oral administration often include binding agents (for example syrups, acacia, gelatin, tragacanth, polyvinylpyrrolidone, sodium lauryl sulphate, pregelatinized maize starch, hydroxypropyl methylcellulose, starches, modified starches, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone and sodium alginate), disintegrants (such as starch and preferably, potato or tapioca starch, alginic acid and certain complex silicates, polyvinylpyrrolidone, gelatin, acacia, sodium starch glycollate, microcrystalline cellulose, crosscarmellose sodium, crospovidone, hydroxypropyl methylcellulose and hydroxypropyl cellulose), lubricating agents (such as magnesium stearate, sodium lauryl sulfate, talc, silica polyethylene glycol waxes, stearic acid, palmitic acid, calcium stearate, carnuba wax, hydrogenated vegetable oils, mineral oils, polyethylene glycols and sodium stearyl fumarate) and fillers (including high molecular weight polyethylene glycols, lactose, calcium phosphate, glycine magnesium stearate, starch, rice flour, chalk, gelatin, microcrystalline cellulose, calcium sulphate, and lactitol). Such preparations may also include preservative agents and anti-oxidants.

Liquid compositions for oral administration may be in the form of, for example, emulsions, syrups, or elixirs, or may be presented as a dry product for reconstitution with water or other suitable vehicle before use. Such liquid compositions may contain conventional additives such as suspending agents (e.g. syrup, methyl cellulose, hydrogenated edible fats, gelatin, hydroxyalkylcelluloses, carboxymethylcellulose, aluminium stearate gel, hydrogenated edible fats) emulsifying agents (e.g. lecithin, sorbitan monooleate, or acacia), aqueous or non-aqueous vehicles (including edible oils, e.g. almond oil, fractionated coconut oil) oily esters (for example esters of glycerine, propylene glycol, polyethylene glycol or ethyl alcohol), glycerine, water or normal saline; preservatives (e.g. methyl or propyl p-hydroxybenzoate or sorbic acid) and conventional flavouring, preservative, sweetening or colouring agents. Diluents such as water, ethanol, propylene glycol, glycerin and combinations thereof may also be included.

Other suitable fillers, binders, disintegrants, lubricants and additional excipients are well known to a person skilled in the art. In some embodiments, compositions include one or more of mucin, antioxidants, reductants, or redox-active compound (e.g., to protect bacteria).

In some embodiments, the bacteria are spray-dried. In other embodiments, bacteria are re-suspended in an oil phase and are encased by at least one protective layer, which is water-soluble (water-soluble derivatives of cellulose or starch, gums or pectins).

In some embodiments, compositions are provided as a nutritional or dietary supplement. In some embodiments, the supplement is provided as a powder or liquid suitable for adding by the consumer to a food or beverage. For example, in some embodiments, the dietary supplement can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food.

The dietary supplement may comprise one or more inert ingredients, especially if it is desirable to limit the number of calories added to the diet by the dietary supplement. For example, the dietary supplement may also contain optional ingredients including, for example, herbs, vitamins, minerals, enhancers, colorants, sweeteners, flavorants, inert ingredients, and the like. For example, the dietary supplement may contain one or more of the following: asorbates (ascorbic acid, mineral ascorbate salts, rose hips, acerola, and the like), dehydroepiandosterone (DHEA), Fo-Ti or Ho Shu Wu (herb common to traditional Asian treatments), Cat's Claw (ancient herbal ingredient), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, zinc, and the like. Such optional ingredients may be either naturally occurring or concentrated forms.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In some embodiments, compositions are provided as nutritional supplements (e.g., energy bars or meal replacement bars or beverages). The nutritional supplement may serve as meal or snack replacement and generally provide nutrient calories. In some embodiments, the nutritional supplements provide carbohydrates, proteins, and fats in balanced amounts.

Sources of protein to be incorporated into the nutritional supplement are any suitable protein utilized in nutritional formulations and can include whey protein, whey protein concentrate, whey powder, egg, soy flour, soy milk soy protein, soy protein isolate, caseinate (e.g., sodium caseinate, sodium calcium caseinate, calcium caseinate, potassium caseinate), animal and vegetable protein and mixtures thereof. In certain embodiments, the protein is a combination of whey protein concentrate and calcium caseinate. These proteins have high biological value; that is, they have a high proportion of the essential amino acids. See Modern Nutrition in Health and Disease, eighth edition, Lea & Febiger, publishers, 1986, especially Volume 1, pages 30-32.

The nutritional supplement can also contain other ingredients, such as one or a combination of other vitamins, minerals, antioxidants, fiber and other dietary supplements (e.g., protein, amino acids, choline, lecithin, omega-3 fatty acids). Selection of one or several of these ingredients is a matter of formulation, design, consumer preference and end-user. Guidance to the amounts or ingredients can be provided by the U.S. RDA doses for children and adults. Further vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulfate; vitamin A; vitamin C; inositol; potassium iodide.

Flavors, coloring agents, spices, nuts and the like can be incorporated into the product. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate.

Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product.

Preservatives may also be added to the nutritional supplement to extend product shelf life. Preferably, preservatives such as potassium sorbate, sodium sorbate, potassium benzoate, sodium benzoate or calcium disodium EDTA are used.

In some embodiments, the nutritional supplement contains natural or artificial (preferably low calorie) sweeteners, e.g., saccharides, cyclamates, aspartamine, aspartame, acesulfame K, and/or sorbitol. Such artificial sweeteners can be desirable if the nutritional supplement is intended to be consumed by an overweight or obese individual, or an individual with type II diabetes who is prone to hyperglycemia.

The nutritional supplement can be provided in a variety of forms, and by a variety of production methods. In one embodiment, to manufacture a food bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein.

In some embodiments, compositions are provided as food products, prepared food products, or foodstuffs comprising prebiotic and bacteria as described above. For example, in some embodiments, beverages and solid or semi-solid foods are provided. These forms can include, but are not limited to, beverages (e.g., soft drinks, milk and other dairy drinks, and diet drinks), baked goods, puddings, dairy products, confections, snack foods, or frozen confections or novelties (e.g., ice cream, milk shakes), prepared frozen meals, candy, snack products (e.g., chips), soups, spreads, sauces, salad dressings, prepared meat products, cheese, yogurt and any other fat or oil containing foods, and food ingredients (e.g., wheat flour).

### EXAMPLES

### Example 1

### A Synbiotic to Protect Against Ethanol-Induced Gut-Liver-Brain Injury in Mice

This Examples describes procedures to test a probiotic blend of L. reuteri and L. plantarum, with either potato starch or green banana flour, to protected against ethanol induced gut-liver-brain injury in a chronic-binge mouse model.

### The Chronic-Binge Mouse Model:

Female C57BL/6J mice, 8-10 weeks old
Fed pair-food (PF) for 5 days, then 5% ethanol (ETOH) for 10 days
Mice gavaged orally daily probiotic blend, synbiotic, or saline starting at Day 1 of EtOH
On day 11, mice received oral gavage of EtOH (5 g/kg) or Maltose
Mice were euthanized 6-hr following gavage
Intestine and liver dissected
   Probiotic Blend: Lactobacillus reuteri 3613 (1×109 CFU). Lactobacillus plantarum 276 (5×109 CFU)
   Prebiotic: Potato starch or green banana flour (20% wt/v)
   Synbiotic: probiotic blend+prebiotic

### Results:

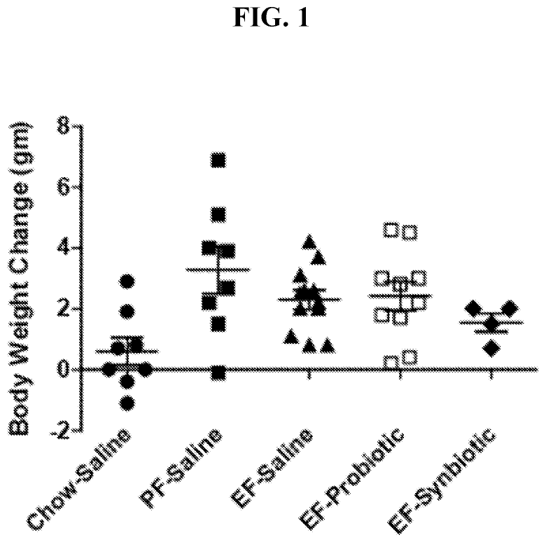

The results, shown in FIGS. 1-9, showed the following: i) the Probiotic blend and synbiotic are well-tolerated in mice; ii) the Probiotic and synbiotic protect against bacterial overgrowth induced by ethanol exposure in mice; iii) the Synbiotic protects against ethanol-induced hepatic steatosis in mice; iv) the Synbiotic protects intestinal tight junctional proteins in the proximal colon of mice; v) the Synbiotic confers potential protection against ethanol-induced oxidative stress in the proximal colon by upregulating the mRNA expression of SOD2; vi) the Synbiotic protects against microglial activation and neuroinflammation.

FIG. 1 shows body weight changes. All mice tolerated the treatments and supplements well. All mice demonstrated expected weight gain and there were no differences in body weight changes between the groups.

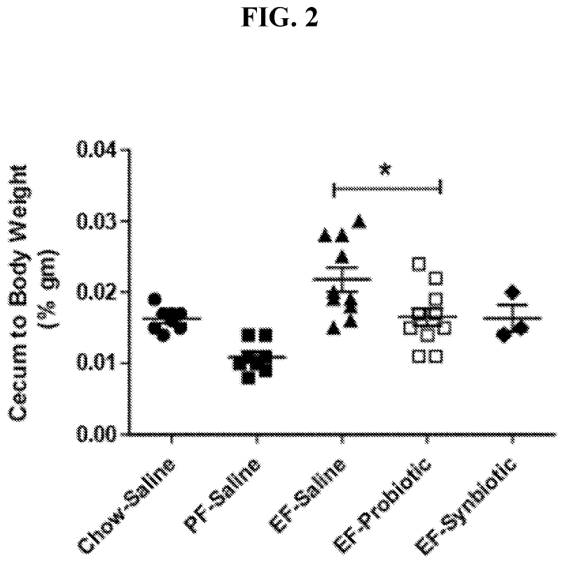

FIG. 2 shows the cecum weight-to-body weight results. Germ-free and/or antibiotic treated (dysbiotic) mice phenotypically have an enlarged cecum. Therefore, we measured the weight of mouse cecum relative to body weight as a surrogate measure for gut dysbiosis. As expected, we found cecum-to-body weight to be highest in the ethanol-saline exposed mice. Mice supplemented with the probiotic blend or synbiotic had a reduced cecum: BW ratio (*P<0.05), similar to chow-fed mice.

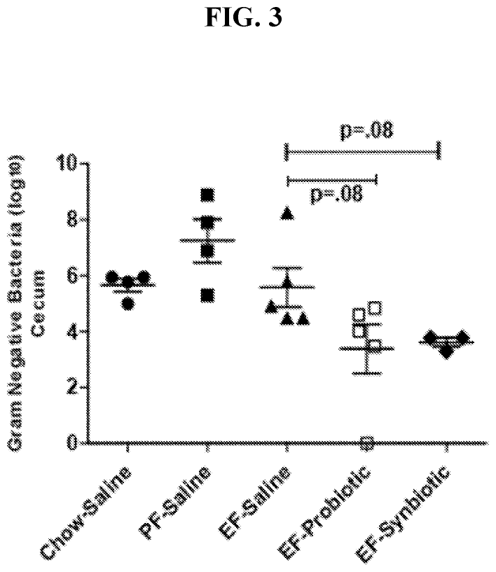

FIG. 3 shows gram negative bacteria in cecum. Ethanol exposure is known to deplete phyla Bacteroidetes and Firmicutes and induce Proteobacteria, which contains gram negative bacteria. Here we tested for overgrowth of gram negative bacteria by selective agar plating with mouse cecal contents. We found that the pair-fed mice, a diet that is high in fat and low in fiber, had the highest presence of gram negative bacteria followed by the ethanol-saline exposed mice. The mice supplemented with probiotic blend or synbiotic had the lowest presence of gram negative bacteria in the cecum (p=0.08).

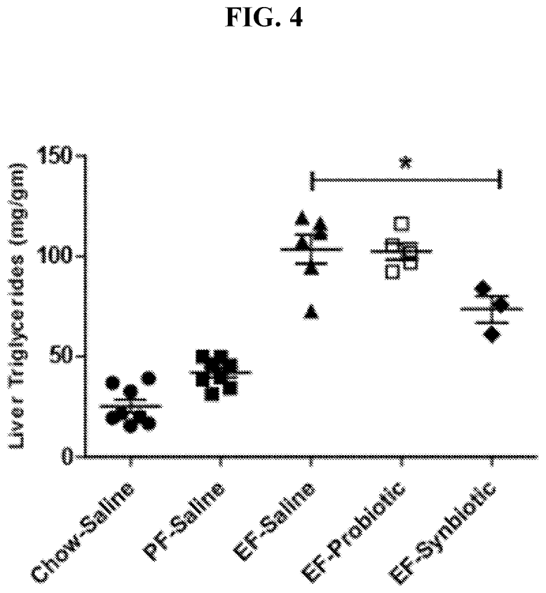

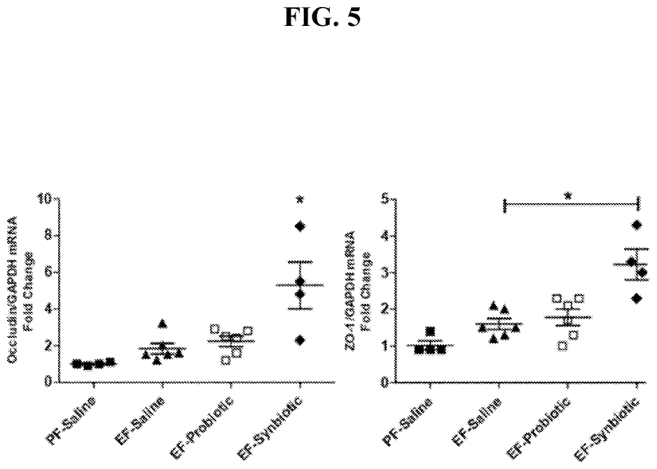

FIG. 4 shows liver triglyceride levels. The ethanol-feeding model induces hepatic steatosis, therefore we looked for differences in liver triglyceride between the treatment groups. We found that ethanol induced liver triglyceride levels compared to the chow-fed and pair-fed mice, but ethanol-exposed mice supplemented with the synbiotic had significantly lower liver triglyceride levels compared to the ethanol-saline treated mice (*P<0.05).

FIG. 5 shows the mRNA expression of two tight junctional proteins, occludin and zonulen occluden (ZO-1) in the proximal colon. Both occludin and ZO-1 mRNA relative expression was significantly higher in the ethanol exposed mice supplemented with the synbiotic (*p<0.05).

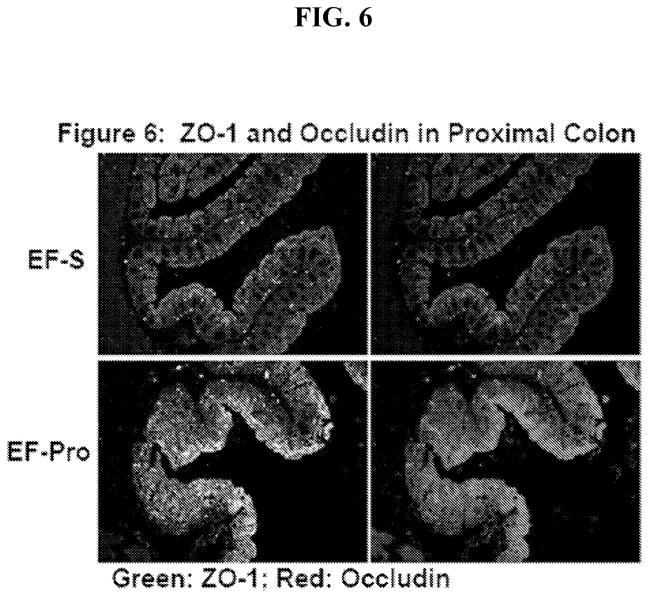

FIG. 6 shows staining results for ZO-1 and Occludin in proximal colon in mice. It was found that ethanol-exposed mice treated with the probiotic blend (EF-pro) had increased staining intensity for ZO-1 (green) and Occludin (red) in the proximal colon compared to mice exposed to ethanol/saline (EF-S).

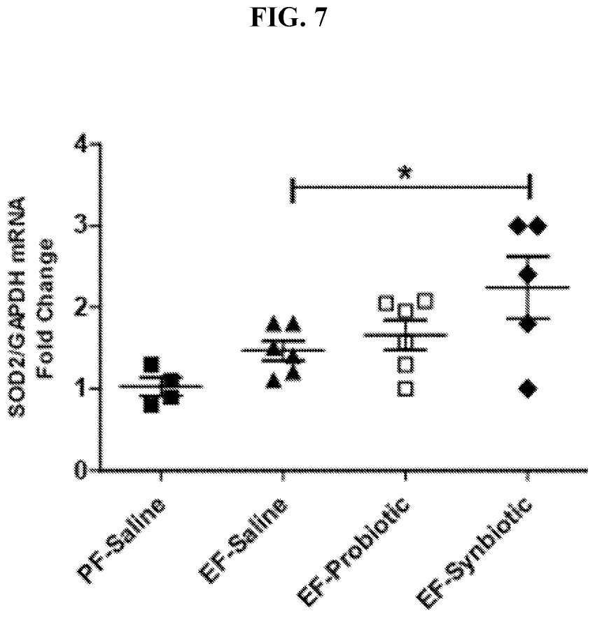

FIG. 7 shows the tested probiotic/synbiotic supplementation in ethanol treated mice increased mRNA expression levels of superoxide dismutase 2(SOD2 ). This is important as ethanol metabolism is known to induce reactive oxygen species and cause oxidative stress, while SOD2 clears mitochondrial reactive oxygen species (ROS) and thus plays an anti-apoptotic role against oxidative stress. We tested for the mRNA expression of SOD2 in the proximal colon and found that the synbiotic induced SOD2 during ethanol exposure compared to the ethanol-saline group (*p<0.05).

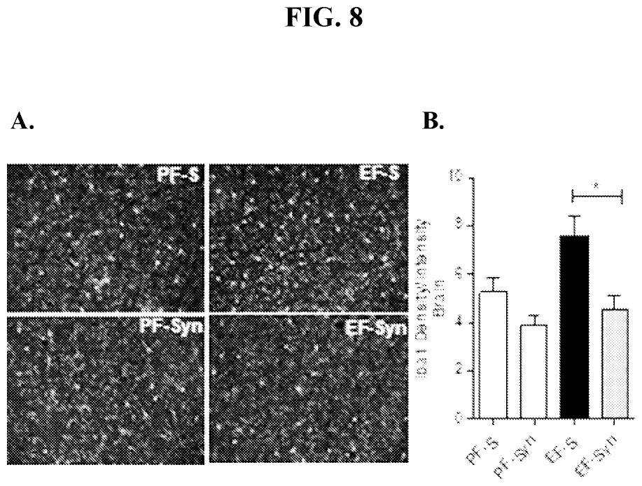

FIG. 8A shows CNS exposure to ethanol deregulates neuroimmune signaling triggering microglial activation. FIG. 8A data shows chronic-binge ethanol exposure induced microglial activation (ionized calcium binding adaptor molecule 1; Iba1) in mice, which was mitigated in mice supplemented daily with a neurotransmitter-targeted synbiotic. FIG. 8B shows Iba1 (green) intensity staining semi-quantification. *p<0.05.

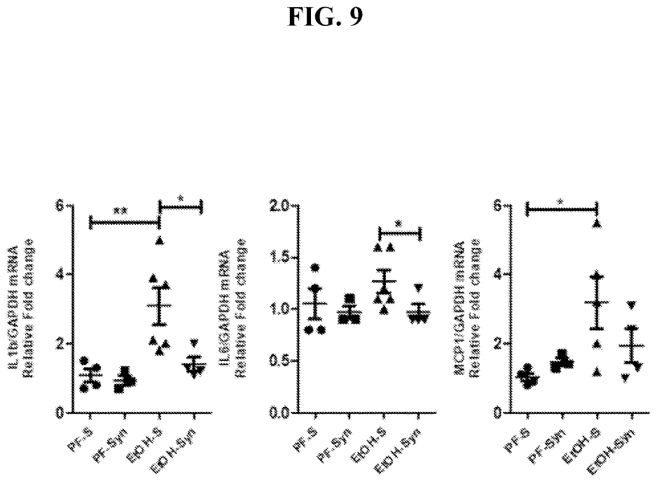

It is known that, after becoming activated, microglia produce and secrete a plethora of inflammatory molecules that when in excess in the parenchyma further contributes to neuronal damage. Ethanol-induced microglial activation (FIG. 8) coincided with increased mRNA expression of proinflammatory cytokines and chemokine, and synbiotic supplementation protected against this occurrence, as shown in this figure (FIG. 9). *p<0.05.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A system comprising:
a) an ingestible capsule;
b) a prebiotic;
c) at least one bacteria selected from:
i) Lactobacillus reuteri, and
ii) Lactobacillus plantarum.

2. The system of claim 1, wherein said at least one bacteria comprises both said Lactobacillus reuteri, and Lactobacillus plantarum.

3. The system of claim 1, wherein said at least one bacteria comprises Lactobacillus reuteri, and said Lactobacillus reuteri comprises Lactobacillus reuteri 3613.

4. The system of claim 1, wherein said at least one bacteria comprises Lactobacillus plantarum, and said Lactobacillus plantarum comprises Lactobacillus plantarum 276.

5. The system of claim 1, wherein said at least one bacteria and said prebiotic are present inside said capsule.

6. The system of claim 1, wherein said capsule comprises a vegetable-based capsule or gelatin-based capsule.

7. The system of claim 1, wherein said at least one bacteria and said prebiotic are mixed and in the form of a powder, which is optionally in said capsule.

8. The system of claim 1, wherein said prebiotic comprises resistant starch.

9. The system of claim 8, wherein said resistant starch is selected from the group consisting of: green banana flour, potato starch, and rolled oats.

10. The system of claim 1, wherein said prebiotic comprises NUBANA RS65G Green Banana Flour.

11. The system of claim 1, wherein said prebiotic comprises oligosaccharides.

12. The system of claim 11, wherein said oligosaccharides are selected from the group consisting of: galactooligosaccharides, fructooligosaccharides, and human milk oligosaccharides.

13. The system of claim 1, wherein said at least one bacteria and said prebiotic are present inside said capsule, and wherein 2-15 mg of said at least one bacteria are present in said capsule.

14. The system of claim 13, wherein 20-700 mg of said prebiotic is present in said capsule.

15. A method of treating or preventing a brain disorder comprising:
administering a composition to a subject, or providing said composition to said subject such that said subject administers said composition to themselves,
wherein said composition comprises:
a) a prebiotic, and
b) at least one bacteria selected from Lactobacillus reuteri, and Lactobacillus plantarum, and
wherein said subject has, or is at risk of, a brain disorder.

16. The method of claim 15, wherein said brain disorder is selected from the group consisting of: neuroinflammation, a neurological disorder, a neuropsychiatric disorder, and a behavioral disorder.

17. The method of claim 15, wherein said brain disorder is selected from the group consisting of: alcohol use disorder, drug addiction, eating disorder, depression, anxiety, autism spectrum disorder, and Alzheimer's disease.

18. The method of claim 15, wherein said composition is in the form of a tablet, suppository, or capsule.

19. The method of claim 15, wherein said administering or administers is orally or rectally.

20. The method of claim 15, wherein said at least one bacteria comprises both said Lactobacillus reuteri, and Lactobacillus plantarum.

21. The method of claim 15, wherein said at least one bacteria comprises Lactobacillus reuteri, and said Lactobacillus reuteri comprises Lactobacillus reuteri 3613.

22. The method of claim 15, wherein said at least one bacteria comprises Lactobacillus plantarum, and said Lactobacillus plantarum comprises Lactobacillus plantarum 276.

23. The method of claim 15, wherein said at least one bacteria and said prebiotic are present inside a capsule.

24. The method of claim 23, wherein said capsule comprises a vegetable-based capsule or gelatin-based capsule.

25. The method of claim 15, wherein said at least one bacteria and said prebiotic are mixed and in the form of a powder, which is optionally present in said capsule.

26. The method of claim 15, wherein said prebiotic comprises resistant starch.

27. The method of claim 26, wherein said resistant starch is selected from the group consisting of: green banana flour, potato starch, and rolled oats.

28. The method of claim 15, wherein said prebiotic comprises NUBANA RS65G Green Banana Flour.

29. The method of claim 15, wherein said prebiotic comprises oligosaccharides.

30. The method of claim 29, wherein said oligosaccharides are selected from the group consisting of: galactooligosaccharides, fructooligosaccharides, and human milk oligosaccharides.

31. The method of claim 15, wherein at least part of said composition is present inside a capsule, and wherein 2-15 mg of said at least one bacteria are present in said capsule.

32. The method of claim 31, wherein 20-700 mg of said prebiotic is present in said capsule.

33. The method of claim 31, wherein at least part of said composition is present inside four to six capsules, and wherein 2-15 mg of said at least one bacteria is present in each of said four to six capsules.

34. The method of claim 33, wherein 20-700 mg of said prebiotic is present in each of said four to six capsules.

35. The method of claim 34, wherein said administering or said administers provides said subject with said four to six capsules on a first day.

36. The method of claim 35, wherein said administering or said administers is repeated on a second day, and optionally on a third day.

37. The method of claim 15, wherein said subject is a human.
